(19) United States
(12) Patent Application Publication
(10) Pub. No.: US 2026/0260758 A1
(43) Pub. Date: Sep. 3, 2026

(54) APPARATUS, METHOD, AND SYSTEM FOR DETERMINING A RISK LEVEL OF A SOMNAMBULISM EVENT OCCURRENCE AND APPARATUS, METHOD, AND SYSTEM FOR DETECTING A SOMNAMBULISM EVENT

(71) Applicant: Sony Group Corporation, Tokyo (JP)

(72) Inventors: Pablo BARROS, Stuttgart (DE); Anja PFLUG, Stuttgart (DE); Gonzalo BAILADOR DEL POZO, Stuttgart (DE); Serkan ERYILMAZ, Stuttgart (DE); Hugo EMBRECHTS, Stuttgart (DE)

(73) Assignee: Sony Group Corporation, Tokyo (JP)

(21) Appl. No.: 19/158,124

(22) PCT Filed: Mar. 5, 2024
(86) PCT No.: PCT/EP2024/055739

§ 371 Date: Aug. 20, 2025

(30) Foreign Application Priority Data
Mar. 10, 2023 (EP) 23161140.1

(51) Int. Cl. G16H 50/30; A61B 5/00; G16H 50/20

(52) U.S. Cl. CPC G16H 50/30; A61B 5/4812; A61B 5/7267; A61B 5/7275; G16H 50/20

(57) ABSTRACT

An apparatus is provided for determining a somnambulism event risk level score of a user. The apparatus includes interface circuitry configured to receive physiological data of the user generated before a sleep period, and processing circuitry configured to determine a risk level score indicating a likelihood of a somnambulism event during the sleep period based on the physiological data. Another apparatus is provided for detecting the onset of a somnambulism event during a sleep period. This apparatus includes interface circuitry configured to receive movement and/or position measurements generated during the sleep period and the risk level score, and processing circuitry configured to detect the onset of a somnambulism event based on the received measurements and risk level score.

### FIELD

This present disclosure is related to using physiological data for determining a risk level score of a somnambulism event occurrence and detecting the onset of a somnambulism event.

### BACKGROUND

Somnambulism, also known as sleepwalking, is a sleeping disorder that involves getting up and walking around while in a state of sleep. A person experiencing a somnambulism event appears to perform conscious actions or present conscious behavior, such as walking, talking, getting dressed, or moving objects. It is a condition that affects, according to recent research, 29% of children, and 4% of adults. However, given that in most cases the person does not remember the somnambulism event, the numbers could be higher. Somnambulism in adults is usually associated with other sleep disorders or elevated levels of stress and may lead to a reduction in sleep quality. In some cases, the person may even perform dangerous actions, such as leaving home or manipulating heavy or dangerous objects, which may cause direct harm.

No standard treatment exists for somnambulism, as its cause and impact on the person's life vary greatly. While common solutions exist that reduce the harm of somnambulism events, such as increasing environmental safety (closing doors and windows, keeping sharp objects away, etc.), it still does not prevent a disturbance in the person's sleep quality. Anticipated awakening, by identifying specific times of the night when the somnambulism events occur, is one proposed solution. This, however, requires a deep understanding and predictability of somnambulism events. Another solution to address somnambulism and also improve sleep quality is the use of cognitive behavior therapy. Although shown to be effective, it is still difficult to track and identify the somnambulism events reliably, since the person usually does not remember any portion of the event after waking up.

Thus, there is a demand for detecting somnambulism events with precision, in particular the onset of a somnambulism behavior.

### SUMMARY

This demand is met by an apparatus, a method, and a system for determining a somnambulism event risk level of a user before a sleep period of the user and by an apparatus, a method, and a system for detecting the onset of a somnambulism event of a user during the sleep period according to the independent claims. Further embodiments are given in the dependent claims. According to a first aspect, the present disclosure relates to an apparatus for determining a somnambulism event risk level score of a user. The apparatus comprises interface circuitry configured to receive, before a sleep period of the user, physiological data of the user generated before the sleep period. The apparatus further comprises processing circuitry configured to determine a risk level score representing a likelihood that a somnambulism event of the user will occur during the sleep period based on the physiological data generated before the sleep period.

According to a second aspect, the present disclosure relates to a method for determining a somnambulism event risk level score of a user. The method comprises receiving, before a sleep period of the user, physiological data of the user generated before the sleep period. The method further comprises determining a risk level score representing a likelihood that a somnambulism event of the user will occur during the sleep period based on processing the physiological data by a trained machine learning model.

According to a third aspect, the present disclosure relates to an apparatus for detecting the onset of a somnambulism event of a user during a sleep period of the user. The apparatus comprises interface circuitry configured to receive movement and/or position measurements of the user generated during the sleep period of the user. The interface circuitry is also configured to receive a risk level score representing a likelihood that a somnambulism event will occur for the user during the sleep period. The apparatus further comprises processing circuitry configured to detect the onset of a somnambulism event of the user based on the movement and/or position measurements generated during the sleep period and the risk level score. According to a fourth aspect, the present disclosure relates to a method for detecting the onset of a somnambulism event of a user during a sleep period of the user. The method comprises receiving movement and/or position measurements of the user generated during the sleep period. The method also comprises receiving a risk level score representing a likelihood that a somnambulism event will occur for the user during the sleep period. The method further comprises detecting the onset of a somnambulism event of the user based on the movement and/or position measurements generated during the sleep period and the risk level score.

According to a fifth aspect, the present disclosure relates to a system for detecting the onset of a somnambulism event of a user during a sleep period of the user. The system comprises a first apparatus for determining a somnambulism event risk level score of a user. The first apparatus comprises interface circuitry configured to receive, before the sleep period, physiological data of the user generated before the sleep period and processing circuitry configured to determine a risk level score representing a likelihood that a somnambulism event of the user will occur during the sleep period based on the physiological data generated before the sleep period. The system further comprises a second apparatus for detecting the onset of a somnambulism event of the user during the sleep period. The second apparatus comprises interface circuitry configured to receive movement and/or position measurements of the user generated during the sleep period and the risk level score from the first apparatus. The second apparatus further comprises processing circuitry configured to detect the onset of a somnambulism event of the user based on the movement and/or position measurements and the risk level score.

According to a sixth aspect, the present disclosure relates to a system for determining a somnambulism event risk level score of a user before a sleep period of the user. The system comprises a first apparatus for detecting the onset of a somnambulism event of the user while the user was sleeping. The first apparatus comprises interface circuitry configured to receive sleeping physiological data of the user generated while the user was sleeping. The sleeping physiological data includes movement and/or position measurements. The first apparatus further comprises processing circuitry configured to detect the onset of a somnambulism event of the user based on the sleeping physiological data. The system further comprises a second apparatus for determining a somnambulism event risk level score of a user. The second apparatus comprises interface circuitry configured to receive physiological data of the user generated while the user was awake, and, from the first apparatus, labeled sleeping physiological data of the user generated while the user was sleeping and comprising a label of whether or not the sleeping physiological data corresponds to a somnambulism event. The second apparatus further comprises processing circuitry configured to determine a risk level score representing a likelihood that a somnambulism event of the user will occur during the sleep period based on the physiological data generated while the user was awake and the labeled sleeping physiological data.

### BRIEF DESCRIPTION OF THE FIGURES

Some examples of apparatuses and/or methods will be described in the following by way of example only, and with reference to the accompanying figures, in which

FIG. 1 schematically illustrates an exemplary apparatus for determining a somnambulism event risk level score of a user;

FIG. 2 schematically illustrates another exemplary apparatus for determining a somnambulism event risk level score of a user;

FIG. 3 schematically illustrates an exemplary apparatus for detecting the onset of a somnambulism event of a user during a sleep period of the user;

FIG. 4 schematically illustrates another exemplary apparatus for detecting the onset of a somnambulism event of a user during a sleep period of the user;

FIG. 5 illustrates a flow chart of sleep period phases related to a somnambulism event that may be detected and classified by the apparatuses of FIGS. 3 and 4;

FIG. 6 schematically illustrates a system for determining a somnambulism event risk level score of a user before a sleep period of the user and for detecting the onset of a somnambulism event of the user during the sleep period at least in part based on the risk level score.

FIG. 7 schematically illustrates a system for detecting a somnambulism event of a user during a sleep period and determining a somnambulism event risk level score of the user at least in part based on detection of the somnambulism event.

FIG. 8 illustrates a flow chart of an exemplary method for determining a somnambulism event risk level score of a user; and

FIG. 9 illustrates a flow chart of an exemplary method for detecting the onset of a somnambulism event of a user during a sleep period of the user;

### DETAILED DESCRIPTION

Some examples are now described in more detail with reference to the enclosed figures. However, other possible examples are not limited to the features of these embodiments described in detail. Other examples may include modifications of the features as well as equivalents and alternatives to the features. Furthermore, the terminology used herein to describe certain examples should not be restrictive of further possible examples.

Throughout the description of the figures same or similar reference numerals refer to same or similar elements and/or features, which may be identical or implemented in a modified form while providing the same or a similar function. The thickness of lines, layers and/or areas in the figures may also be exaggerated for clarification.

When two elements A and B are combined using an “or”, this is to be understood as disclosing all possible combinations, i.e. only A, only B as well as A and B, unless expressly defined otherwise in the individual case. As an alternative wording for the same combinations, “at least one of A and B” or “A and/or B” may be used. This applies equivalently to combinations of more than two elements.

If a singular form, such as “a”, “an” and “the” is used and the use of only a single element is not defined as mandatory either explicitly or implicitly, further examples may also use several elements to implement the same function. If a function is described below as implemented using multiple elements, further examples may implement the same function using a single element or a single processing entity. It is further understood that the terms “include”, “including”, “comprise” and/or “comprising”, when used, describe the presence of the specified features, integers, steps, operations, processes, elements, components and/or a group thereof, but do not exclude the presence or addition of one or more other features, integers, steps, operations, processes, elements, components and/or a group thereof.

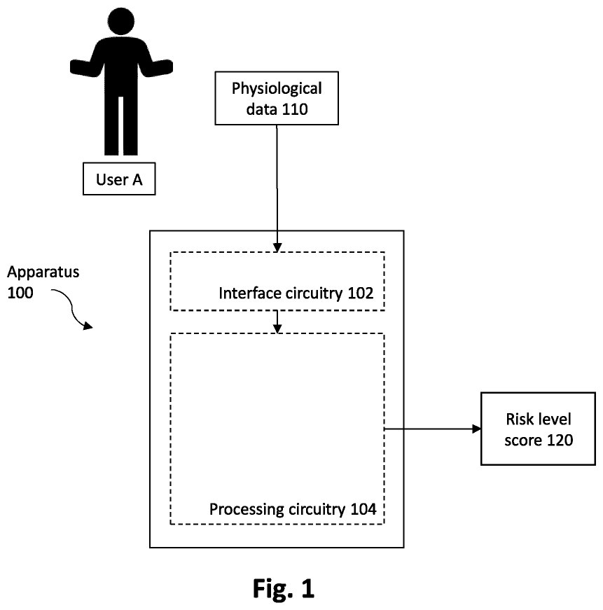

FIG. 1 schematically illustrates an apparatus 100 for determining a somnambulism event risk level score 120 of a user.

Somnambulism, also known as sleepwalking, is a sleeping disorder leading to abnormal behavior during sleep. Somnambulism events involve a person getting up and walking around while in a state of sleep, which may lead the person to be vulnerable to accidents or injuries. A somnambulism event may end after the person has woken up or has returned to a resting position and is no longer exhibiting movements required for getting up. If the person has returned to bed and the somnambulism event has finished, the person may continue sleeping and a cycling between a rapid-eye movement (REM) sleep state and non-REM (NREM) sleep state may continue, as is the case in an undisturbed and uninterrupted period of sleep.

Through studying somnambulism events, a link has been established between stress levels experienced by a person and the likelihood that the person will experience a somnambulism event during a sleep period in the near future. Stress levels of a person may be detected in numerous ways. For example, a person experiencing multiple stressful moments over the course of a day or chronic stress may exhibit directly recognizable symptoms. Other means of recognizing stress may require recording physiological data of the person with sensors over an extended time period. Since somnambulism events are highly associated with daily stressors, tracking physiological data of the person could allow a prediction of the likelihood of a somnambulism event occurring during the night. Combining such a prediction with a method of detection of a somnambulism event may enable both the precise identification and efficient mitigation of somnambulism events.

As will be evident in the following description, the apparatus 100 allows to determine the risk level score 120, which represents a likelihood that a somnambulism event will occur for a user during a sleep period in the near future. The risk level score 120 may be expressed in terms of a probability. The risk level score 120 may also be expressed as a selection of pre-defined categories that are labeled to express a risk level, such as low risk, moderate risk, or high risk. The risk level score 120 may include a description providing further details related to the risk level, such as how the somnambulism event is expected to occur. For example, the description may include an expected time of occurrence for the event, an expected duration of the event, whether multiple events are expected, and expected behaviors of the user during the event (if not interrupted). The risk level score 120 may provide any combination of such features to the user before a sleep period of the user. The risk level score 120 may provide the user with information to adjust an environmental setting for sleeping to ensure safety.

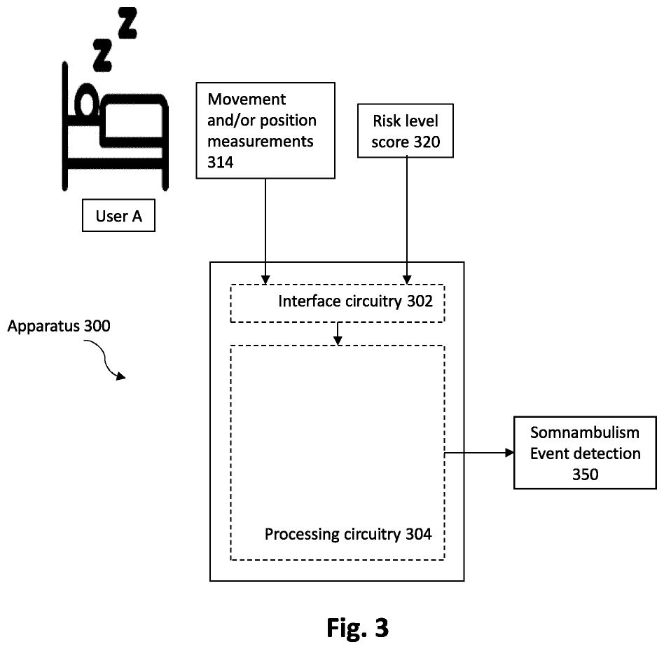

The risk level score 120 provided through multiple iterations may also enable the user to better understand possible causes of somnambulism events particular to the user. More specifically, the user may be made aware of a particular lifestyle or particular habits that may be changed or adapted in the case of a high risk. Obtaining a risk level score every day before each upcoming sleep period may especially improve the user's understanding of possible causes. Such risk level scores recorded daily or at least incrementally over time may also aid medical professionals and researchers to better understand the causes of somnambulism events and how these causes range for different people. In particular, analyzing the fluctuation of risk levels over time may provide insight into possible causes of somnambulism events. Also, described later in FIGS. 3 to 6, another apparatus for detecting the onset of a somnambulism event may complement the features of the apparatus 100 to obtain precise feedback. Together, the two apparatuses may enable obtaining an understanding of specific characteristics of somnambulism events that are particular for each user.

The apparatus 100 for determining the risk level score 120 comprises interface circuitry 102 configured to receive physiological data 110 of the user before the user's sleep period. The apparatus 100 further comprises processing circuitry 104 coupled to the interface circuitry 102. The processing circuitry 104 may be a single dedicated processor, a single shared processor, or a plurality of individual processors, some of which or all of which may be shared. Alternatively, the processing circuitry 104 may be a digital signal processor (DSP) hardware, an application specific integrated circuit (ASIC), a neuromorphic processor or a field programmable gate array (FPGA). The processing circuitry 104 may optionally be coupled to, e.g., read only memory (ROM) for storing software, random access memory (RAM) and/or non-volatile memory. Optionally, the apparatus 100 may comprise further circuitry.

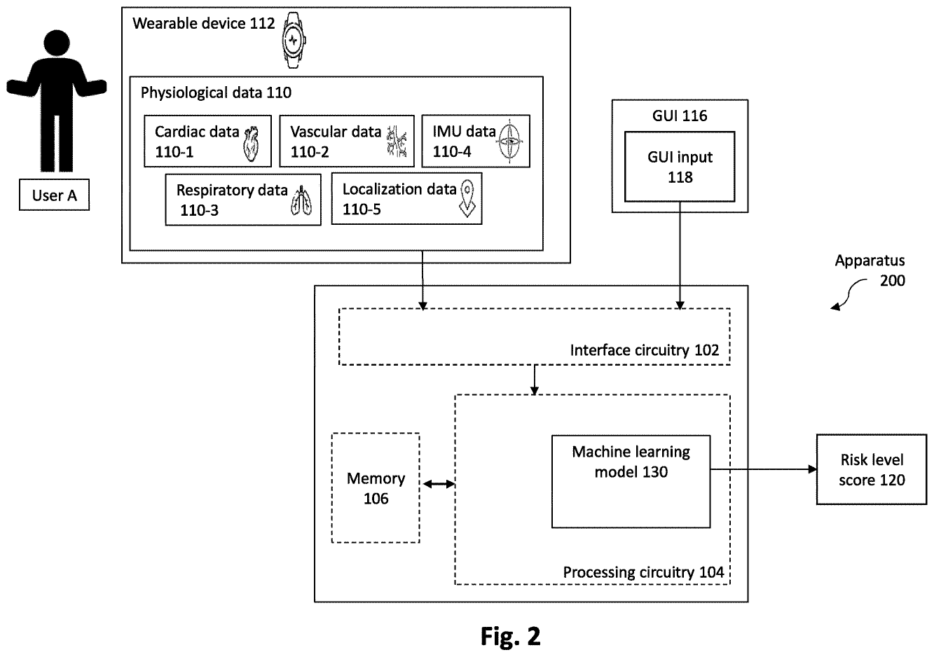

The processing circuitry may be configured to determine the risk level score 120 based on multiple forms of data within the physiological data 110. For example, the physiological data 110 may relate to bodily functions of the user, which may include (shown in FIG. 2) cardiac data 110-1 related to the person's heart activity, vascular data 110-2 related to the person's blood vessel activity, and respiratory data 110-3 related to the person's breathing activity with the lungs. Such data measurements may provide direct indications of whether the user has experienced a stressful event or chronic stress. To provide greater context, the physiological data 110 may also include inertia measurement unit (IMU) data 110-4 related to the person's movement in multiple axes and localization data 110-5 related to the location of the person. The different types of data within the physiological data 110 may be analyzed in different combinations and applying different methods to reveal whether the person is experiencing a high level of stress and in what context.

The physiological data 110 may correspond to a user that is awake and active, as depicted by the user A in FIG. 1. The processing of the physiological data 110 may be based on a pre-specified time period of data collection directly before the sleep period. The time period may correspond to a time while the user was awake and active and optionally while the user was sleeping during previous sleep periods. As a typical example, the time period of data collection may be pre-specified to be one day or one week, but may range from a scale of minutes, hours, days, weeks, months, or even years. The physiological data 110 may have been either continuously recorded throughout the pre-specified time period or recorded in multiple intervals. The physiological data 110 within a first portion of the time period may be given greater weight compared to the physiological data 110 within a second portion. For example, the user is more likely to still be affected by a stressful event occurring during a more recent time period. If the user experienced high stress levels in the last three hours, it may determine the risk level score 120 to be higher compared to the user experiencing the same stress levels in a three-hour span that occurred two days ago.

In general, the pre-specified time period may be used to enable an efficiency in processing, since it is unlikely that physiological data recorded in a time span occurring multiple months before will affect the risk level score 120 in a way that would not be identified in more recently recorded physiological data. Such a pre-specified time period of data collection for the physiological data 110 may be customized according to a user input. For example, if the user is aware of experiencing a stressful event, the time period may be extended to include the physiological data 110 measured during the stressful event for determining the risk level score 120. In such an example, if the time period is usually specified to include the physiological data 110 measured during the past single day and the stressful event occurred two days ago, then the user can manually adjust the pre-specified time period to include the stressful event for determining the risk level score 120.

The pre-specified time period may also be automatically adjusted based on one or more thresholds within the physiological data 110. For example, the user may experience an episode of extended heavy breathing under stress, which may surpass a threshold within the respiratory data 110-3. The threshold in the respiratory data 110-3 may be verified with other forms of physiological data. The surpassing of the threshold may cause the data identifying the stress-related event to be included in the physiological data 110 for multiple iterations of determining the risk level score 120. For example, if the pre-specified time period is normally one day and the stressful event occurred two days ago, the pre-specified time period may be extended to two days to include the stressful event. For determining the risk level score 120 a day later, the time period may be extended to three days and continually extended based on the severity of the event.

Selecting the most relevant portions of the physiological data 110, such as choosing the most recently recorded data and adjusting the pre-specified time as needed, may enable the apparatus to perform an efficient processing to provide daily feedback to the user. Further features may allow an easier recording and transfer of data and may enable the apparatus to provide a more accurate risk level score, as described in FIG. 2.

FIG. 2 schematically illustrates another exemplary apparatus 200 for determining the somnambulism event risk level score 120 of a user.

In the apparatus 200, the interface circuitry 102 may be communicatively connectable to a wearable device 112 and configured to receive the physiological data 110 from the wearable device 112. The wearable device may be worn by a user that is awake and active, as depicted by the user A in FIG. 2. In general, the user may wear one or more wearable devices equipped with sensors for the measurement of physiological data, which may be immediately streamed upon measurement to the apparatus 200 or transmitted to the apparatus 200 incrementally. The wearable device 112 may take multiple different forms to record measurements of the physiological data 110.

For example, the wearable device 112 may be a smartwatch equipped with sensors configured to record a heart rate of the user over time. The heart rate may be recorded by means of a blood volume pulse measurement. For example, the smartwatch may be equipped with an LED on its inner side that flashes light signals, as well as light-sensitive photodiodes to detect changes between light signals based on volume changes in capillaries above the user's wrist. The wearable device 112 may be another sensor to be worn by a fingertip or another part of the body. For example, the wearable device 112 may be a photoplethysmogram configured to measure a heart rate and/or blood volume pulse. The wearable device 112 may also be configured to measure characteristics of the user's skin, such as a skin temperature or electrodermal activity related to continuous variation in the electrical characteristics of the skin. Such measurements may be processed to provide context to the physiological data related to a heart rate or blood volume pulse measurements. The wearable device 112 may also be equipped with an accelerometer and/or gyroscope that provides context of how much the user is moving and what may be causing a raised or decreased heart rate, among other trends in the data.

The processing circuitry 104 may receive portions of the physiological data 110 that have already been processed by the wearable device 112. For example, the respiratory data 310-3 may be derived from data related to the heart rate of the user or from optical measurements reflected from the user's skin. If the wearable device receives such data, it may already process the data to provide portions of the respiratory data 310-3. Alternatively or additionally, the processing circuitry 104 may receive raw sensor data from the wearable device 112 and process the raw sensor data to derive portions of the physiological data 110. Once the raw sensor data has been processed and transformed to the physiological data 110 by the wearable device 112 and/or by the processing circuitry 104, the processing circuitry 104 may determine the risk level score 120 based thereon.

The processing circuitry 104 may further comprise a memory 106. The memory may be configured to store the physiological data 110 after being received by the interface circuitry 102 and used by the processing circuitry 104 to determine the risk level score 120. Thus, the physiological data 110 may be stored as previously measured physiological data 110 and may be saved as a past physiological dataset. Over many iterations of determining the risk level score 120, the memory may accumulate many past physiological datasets corresponding to the user. A past physiological dataset may be an isolated set of physiological data that corresponds to a specific user and to a specific time period, such as one day or one week, and is labeled whether it corresponds to a somnambulism event or not. Such past physiological datasets may provide a basis for the apparatus 200 to identify certain characteristics that indicate a greater likelihood of a somnambulism event. Upon receiving the (current) physiological data 110 of the user, the apparatus 200 may be configured to determine the risk level score 120 for the user based on a pattern matching analysis that searches the physiological data 110 for patterns that may be similar to the indicative characteristics identified in the past physiological datasets.

Each past physiological dataset may comprise data that either does or does not correspond to a past somnambulism event. In order to identify characteristics within the physiological data 110 that may be more likely to lead to a somnambulism event, the processing circuitry 104 may perform a pattern recognition analysis comparing multiple past physiological datasets that did not lead to a somnambulism event with multiple past physiological datasets that did lead to a past somnambulism event.

The pattern recognition analysis may identify characteristics that are found to have a higher likelihood of leading to a somnambulism event occurrence for the respective user or any user shortly after the corresponding data was measured. For example, a past physiological dataset that led to a somnambulism event occurrence may show a higher average heart rate or respiratory rate, or specific time periods during which the heart rate or respiratory rate of the respective user was exceptionally high. The analysis may also identify such characteristics across multiple types of physiological data. For example, certain forms of data, such as IMU data 110-4 or localization data 110-5, may provide more precise context for other forms of data, such as the cardiac data 110-1, the vascular data 110-2, or the respiratory 110-3 that measure bodily functions more directly. If the heart rate or respiratory rate was very high and the user was not moving, this may be a greater sign of stress that more likely leads to a somnambulism event compared to a dataset showing the user moving quickly by exercising.

Past physiological datasets of both the user and one or more different users may enable the processing circuitry 104 to adjust thresholds accordingly. For example, past physiological datasets based on one or more different users may provide a larger pool of physiological data to create starting values for the thresholds. The user may then provide his own past physiological datasets over time, which may enable adjusting the thresholds to be customized to the user and obtaining more accurate risk level scores. Past physiological datasets that both did or did not lead to a somnambulism event, once provided to the processing circuitry, may enable a pattern recognition analysis more customized toward the user's bodily functions. The pattern recognition analysis may apply appropriate weights to respective datasets. For example, identified characteristics within past physiological datasets of the user can be weighted more in determining thresholds compared to those of one or more different users.

The past physiological datasets may be imported by the apparatus 200 by means of a graphical user interface (GUI) 116. The user may upload a past physiological dataset as part of a GUI input 118 to the apparatus 200. The GUI 116 may also enable the user to label a past physiological dataset according to whether or not it corresponds to a somnambulism event. The apparatus 200 may continually receive further past physiological datasets that either correspond or do not correspond to a past somnambulism event to be included in updated pattern matching analyses. In order to make connections within a large pool of data, particularly with portions recorded over an extended period of time, machine learning models may be implemented. Models with a neural network architecture that apply temporal learning solutions and continual or online learning solutions may perform such pattern matching analyses more efficiently.

The processing circuitry of the apparatus may comprise (use) a machine learning model 130, which may be trained to perform the pattern matching analyses. The machine learning model 130 may be trained by ground truth information including multiple past physiological datasets of the user and/or one or more different users. The training may enable the machine learning model 130 to identify characteristics of the physiological data 110 that are more likely to lead to a somnambulism event for a respective user and to determine the risk level score 120.

The machine learning model 130 may comprise an artificial neural network, such as a recurrent neural network (RNN) or a convolutional neural network (CNN). The neural network may be in the form of a representation encoder, which may include but it is not limited to fully connected layers, convolutional-base encoders, graph-based representations, and neural field encoders. The representational encoder may be configured to encode an input comprising the physiological data 110 into a compressed form in a way that captures important features and patterns, while discarding noise and irrelevant information. To enable an efficient processing of the physiological data 110 into a compressed form of data, the machine learning model 130 may be apply various machine learning methods.

For example, the machine learning model 130 may apply temporal representation learning. Temporal representation in machine learning refers to capturing temporal dependencies and patterns in input data. Raw temporal data within a dataset measured over an extended time period may be transformed into a format that is more efficiently processed by a model for a pattern recognition analysis. Temporal representation may apply long-short-term memory (LSTM) connections or gated recurrent units (GRUs), which apply gating mechanisms, or selective filters to modify the flow of information between different parts or layers of a neural network. This may further enable filtering out irrelevant or redundant data to more efficiently capture long-term dependencies in the temporal data.

For example, a temporal dependency in a past physiological dataset may be related to how stressful events or chronic stress affect the user for long period of time, thus affecting many future sleep periods of the user. More specially, it may identify information how a type or severity of stress and multiple variables related thereto may affect each other and how it may affect future sleep periods of the user. With temporal dependencies learned and established, such information may then be identified in future versions of the physiological data 110. In particular, temporal dependencies may affect how the pre-specified time period may be adjusted to determine the risk level score 120. Many variables related to the stressful event, including severity, duration, and instances of repetition may be collectively analyzed with temporal dependencies to determine changes in the risk level score 120. Over time, the machine learning model 130 may receive further past physiological datasets that also comprise data related to the same or a similar stressful event. The temporal dependencies related thereto may then be updated. Identifying and analyzing temporal dependencies related to multiple types of stressful events may enable a more accurate determination of the risk level score 120.

As more and more past physiological datasets are provided to the apparatus 200, searching for temporal dependencies within a large collection of data may also lead to difficulties in processing. The machine learning model 130 may be able to process such large collections of data more efficiently through the use of attention mechanisms. Attention mechanisms are a way to selectively focus on specific parts of input data, rather than processing the entire input data at once. This may allow the machine learning model 130 to dynamically allocate its resources to the most relevant parts of the physiological data 110 and the past physiological datasets, which may improve accuracy and efficiency. For example, with certain characteristics already identified within past physiological datasets that are known to more likely lead to a somnambulism event, the attention mechanisms may enable the machine learning model 130 to start a search within the physiological data 110 for similar or related characteristics and then gradually search more broadly therefrom.

The attention mechanisms may be a soft attention mechanism, which applies learning weights for a weighted sum of an input sequence within input data to compute a context vector. Each of the weights may indicate an importance of each element in the sequence. The attention mechanism may also be a hard attention mechanism, which involves selecting a single input element for learning via a discrete decision. Furthermore, the attention mechanism may be a multi-head attention mechanism, which forms subunits of the learning model into multiple “heads”, each of which learns a different representation of the input sequence by computing its own set of weights. This may allow the machine learning model 130 to manipulate different parts of the input sequence in different ways. The machine learning model 130 may apply different forms of attention mechanisms for an improved efficiency of processing of the physiological data 110 and past physiological datasets.

The machine learning model 130 may also apply continual or online learning solutions. Continual learning, also known as lifelong learning, involves learning from a continuous stream of data, where the data arrives in a sequential and potentially infinite manner. Online machine learning involves learning from incremental data updates without re-training the entire model for each update. While traditional machine learning usually involves a complete re-training of a model when an update with new ground truth information is desired, continual or online learning enables a more efficient approach for multiple update iterations. Applying continual and/or online learning solutions may enable the machine learning model 130 to learn from multiple iterations of the physiological data 110 while retaining the knowledge it has acquired from past physiological datasets. This may the machine learning model 130 to adapt toward a user's change in routines and/or habits, whenever they occur.

The machine learning model 130 may apply continual and/or online learning solutions when receiving further past physiological datasets. In particular, a catastrophic forgetting, where a learning of the new information leads to forgetting previous knowledge, may be avoided. For example, an uncommon characteristic identified in a past physiological dataset may have been identified in a pattern matching analysis. While uncommon, if such an identified characteristic is a reliable indicator of a future somnambulism event, it is important for the machine learning model 130 to maintain knowledge thereof as the pool of past physiological datasets is expanded. If no past physiological dataset provides any further examples of the uncommon characteristic, it may still be reliably identified when it appears. The machine learning model 130 may apply continual and online learning to retain such uncommon characteristics.

In particular, the representational encoder may improve the efficiency of the machine learning model 130 by applying techniques related to transfer learning. In transfer learning, a model trained on a specific task may be repurposed to a different but related task. As such, the knowledge gained from learning a previous task can save time and computational resources to improve the performance of the model for the new task. This may be applied by the machine learning model 130 in the case of identifying new characteristics related to somnambulism events in the physiological data 110 and past physiological datasets. If a new characteristic is identified that is similar enough, then a method of pattern recognition may be adapted from one that has already been established for a more efficient recognition.

Other similar machine learning techniques that may be applied by the machine learning model 130 are elastic weight consolidation and prioritized experience memory. Elastic weight consolidation may enable the machine learning model 130 to learn a new task, such as searching for a newly identified characteristic, without overwriting weights for a previously learned task. In particular, an importance of each weight in a sequence may be computed in a network for the previously learned task and the importance values can be used to adjust the learning rate during a training for a new task. This may help to preserve knowledge acquired by the machine learning model 130 related to the previously learned task.

The machine learning model 130 may also apply prioritized experience learning. Instead of using only the most recently received physiological data 110 to update a model, such data can also be stored in the memory 106, and an update of the machine learning model 130 can incorporate random samples from the past physiological datasets. Such an approach may improve efficiency and prevent overfitting of a model within the machine learning model 130. In particular, a priority value may be assigned to each sample within a series of samples, placing a greater importance on specific samples. Samples with greater importance are then sampled more frequently to be included in model updates. With such a configuration, the most important characteristics found in past physiological datasets may be incorporated into updates more often, enabling a fine-tuning of the machine learning model 130 that further improves efficiency and prevents overfitting.

The machine learning model 130 may be trained by a first step of learning a representation of multimodal inputs, such as different forms of the physiological data 110, and then a second step of learning how the specific representation evolves over time for a specific user. For example, the machine learning model 130 may examine the cardiac data 110-1 of the user and examine how the cardiac data 110-1 evolves over time for the user. This may also be done for cardiac data of multiple different users to provide a comparison. The machine learning model 130 may also be trained by learning both previously mentioned steps simultaneously. The machine learning model 130 may be trained either in a supervised or self-supervised manner, using the past physiological datasets corresponding to a somnambulism event as a training target. The training may apply optimizers, such as stochastic gradient descents, adaptive moment estimation (ADAM), and variations thereof, which may include AdaGrad, AdaDelta, AdaMax, and ADAMW. In general, the machine learning model 130 may apply optimization techniques, such as weight constraints and regularization, activation and connection dropout, and batch normalization.

The user may receive the risk level score 120 for a somnambulism event occurring in the following sleep period according to the physiological data 110. If the user has a means to determine whether a somnambulism event actually occurs or does not occur, then the physiological data 110 may be saved as a past physiological dataset with a label of whether or not a somnambulism event occurred. That particular labeled dataset may then be used as ground truth information in a further iteration of training for the machine learning model 130, which may be applied according to one or more features previously described. Such updates may reinforce a customization of the machine learning model 130 toward the user.

The pool of ground truth information may be expanded more quickly with a data-sharing system. One or more different users may follow the same previously described procedure of saving the (current) physiological data 110 into the memory 106, such that it becomes a stored past physiological dataset. The past physiological dataset may be stored with a corresponding label of whether or not it is associated with a somnambulism event occurrence. Such past physiological datasets of one or more different users may also be shared in a central location, downloaded, and applied as ground truth information to update the training for the machine learning model 130. The past physiological datasets of the one or more different users may have a decreased weighting within the machine learning model 130 for the training to be customized toward the user.

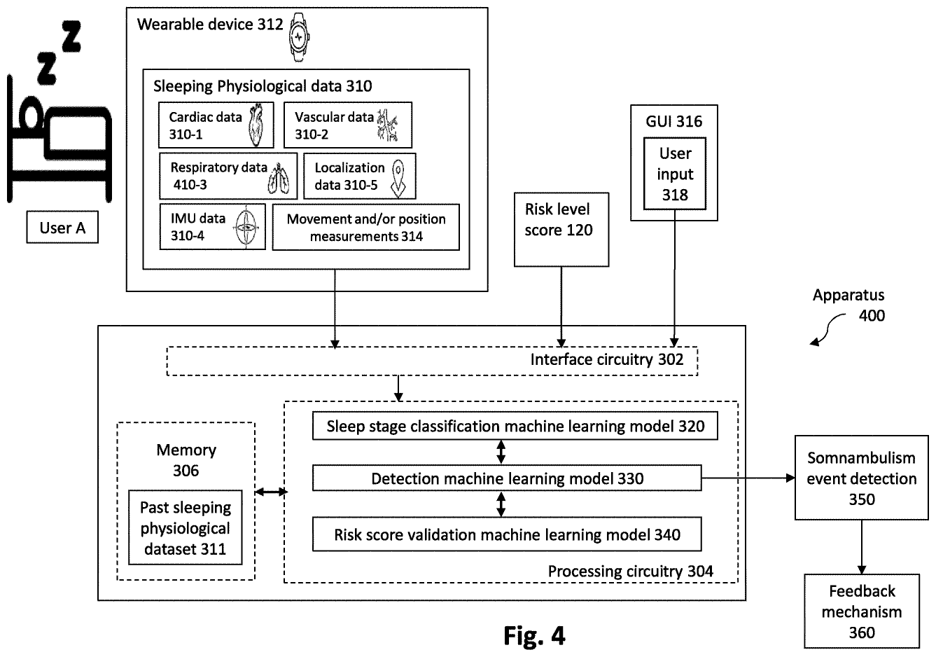

Providing the apparatus 100; 200 with further ground truth information, particularly with such labeled past physiological datasets for the user and one or more different users, may further improve the risk level score determination. An apparatus that is able to precisely detect a somnambulism event and provide data related thereto may greatly complement the features of the apparatus 100; 200 to determine the risk level score 120. Such an apparatus is provided in greater detail in FIGS. 3 and 4.

FIG. 3 schematically illustrates an exemplary apparatus 300 for a detection 350 of the onset of a somnambulism event of a user during a sleep period of the user.

While the apparatus 100; 200 to determine the risk level score 120 serves the user in better understanding the causes of somnambulism events, it is also important for the user to obtain feedback of when the somnambulism events occur. An apparatus for detecting a somnambulism event while the user is sleeping may enable a user to keep track of when events occur and provide feedback for the apparatus 100;2 00 determining the risk level score 120. Furthermore, as previously described, somnambulism events leave those experiencing the event vulnerable to accident or injury. If connected to a feedback device, an apparatus for detection may aid in maintaining safety when a somnambulism event actually occurs. The apparatus 300 in FIG. 3 enables the inclusion of such features for a user that is sleeping, as depicted by the user A. In particular, the apparatus 300 is configured to precisely detect the onset of a somnambulism event.

The apparatus 300 comprises interface circuitry 302 to receive movement and/or position measurements 314 of the user generated during a sleep period of the user. Such measurements may be used to detect the user getting up or supporting his own weight, thus moving from a previous resting position. The movement and/or position measurements 314 may be recorded by an inertial measurement unit (IMU), which may include an accelerometer and/or a gyroscope.

An accelerometer is a sensor that measures changes in acceleration, or changes in motion, along one or more axes. In particular, an accelerometer can detect a change in acceleration that occurs when a user is shifting position to lean upward or stand up. A gyroscope sensor, used to measure angular motion, or angular velocity along one or more axes, may complement an accelerometer to record movement and/or position measurements. The accelerometer and gyroscope may each provide data complementary to each other that enables a detection of more complex movements. The IMU may also include a magnetometer, which may be used to measure the orientation of one or more sensors with respect to the Earth's magnetic field. Measurements by a magnetometer may further complement measurements by the accelerometer and gyroscope by providing a more accurate estimate of a sensor's orientation in three-dimensional space. The magnetometer may particularly be useful for obtaining more detailed information related to particular movements of the user while getting up or after getting up.

The interface circuitry 302 is also configured to receive the risk level score 120 representing a likelihood that a somnambulism event will occur for the user during the sleep period. The risk level score 120 may be provided from the apparatus 100 with the features described in FIG. 1, which may include an expected time, duration, and one or more behaviors expected during the event. The risk level score 120 may also be provided from another apparatus. The apparatus 300 further comprises processing circuitry 304, which may take various forms, including those previously outlined for the apparatus 100 in FIG. 1. The processing circuitry 304 is configured to detect the onset of a somnambulism event of the user based on the movement and/or position measurements 314 generated during the sleep period and based on the risk level score 120. The risk level score 120 may be derived from data measurements of the user before the sleep period, such as the physiological data 110 provided to the apparatus 100; 200.

The processing circuitry 304 is configured to detect the onset of a somnambulism event of the user. The detection of the onset of a somnambulism event may be based on one or more thresholds of the movement and/or position measurements 314. For example, thresholds in the accelerometer and/or gyroscope measurements may be at first set to a value that is determined to be suitable for a wide range of users. Such thresholds may detect a shifting in position or supporting of bodyweight by the user, particularly for getting up from a resting position. The threshold may also be set higher or lower after receiving the risk level score 120. For example, if the risk level score 120 is relatively high, then thresholds may be lowered to encourage an immediate intervention of a somnambulism event at the onset. If the risk level score 120 is relatively low, then thresholds may be lowered to prevent a false positive detection and prevent an unnecessary disturbance of the sleep period. The threshold may also be adapted to a user according to physiological information of the user, which may correspond to the physiological data 110 as previously described for FIGS. 1 and 2. The received physiological data of the user may correspond to the physiological data used to determine the risk level score 120 or a past physiological dataset, as previously described. Such data may relate to one or more stressful events that may indicate a greater likelihood of a somnambulism event occurring in the following sleep period. Thus, such data may provide a more specific input to enable a finer adjustment of the thresholds related to the movement and/or position measurements.

Beyond an input provided before the sleep period of the user, providing sleeping physiological data of the user during the sleep period beyond movement and/or position measurements may further increase the precision of detection. Features related thereto will be described in greater detail in FIG. 4.

FIG. 4 schematically illustrates another exemplary apparatus 400 for detecting a somnambulism event of a user during a sleep period of the user.

The interface circuitry 302 of the apparatus 400 may be communicatively connectable to a wearable device 312, which may be worn by a user that is asleep, as depicted by the user A in FIG. 4. As such, the interface circuitry 302 may be configured to receive sleeping physiological data 310 of the user, or physiological data generated during the sleep period.

The sleeping physiological data 310 may include similar forms of data related to bodily functions compared to the physiological data 110 for the previous apparatus 100; 200. This may include sleeping cardiac data 310-1, sleeping vascular data 310-2, and sleeping respiratory data 310-3. To provide greater context to the bodily functions, the sleeping physiological data 310 may further include sleeping IMU data 310-4 and sleeping localization data 310-5. Each of the datatypes of the sleeping physiological data 310 may be processed into a form more specific to a dataset that relates to a user that is sleeping. For example, sleeping localization data 310-5 may include data related to positions of certain body parts and on a more precise scale of positioning compared to an active user that is awake and often changing location at a much greater pace.

The apparatus 400 may also be configured to detect a somnambulism event on the condition that the user has entered a specific sleep stage. Sleep stages have conventionally been organized into four stages. The first three stages include non-REM, or NREM sleep (N1, N2, and N3), and the fourth stage includes REM sleep. Humans sleeping on a regular schedule exhibit a reliable pattern that includes a cycling between NREM and REM sleep. In particular, sleep begins in a light NREM stage and progresses through deeper NREM stages before an episode of REM sleep begins. While the first two stages (N1 and N2) are considered light sleep stages, the third stage (N3) is considered a deep sleep stage. Somnambulism events are known to usually occur during a deep sleep stage (N3) of a human sleep cycle.

In one embodiment, the apparatus 400 is configured to detect a somnambulism event on the condition that the user is in a deep sleep stage. The deep sleep stage may be detected based on the sleeping physiological data 310 of the user. In particular, the sleeping cardiac data 310-1, the sleeping vascular data 410-2, and the sleeping respiratory data 410-3 may provide data directly related to bodily functions that may be used to detect the deep sleep stage. The sleeping IMU data 310-4 and sleeping localization data 310-5 may also provide complementary data to such data to aid in detecting a deep sleep stage of the user.

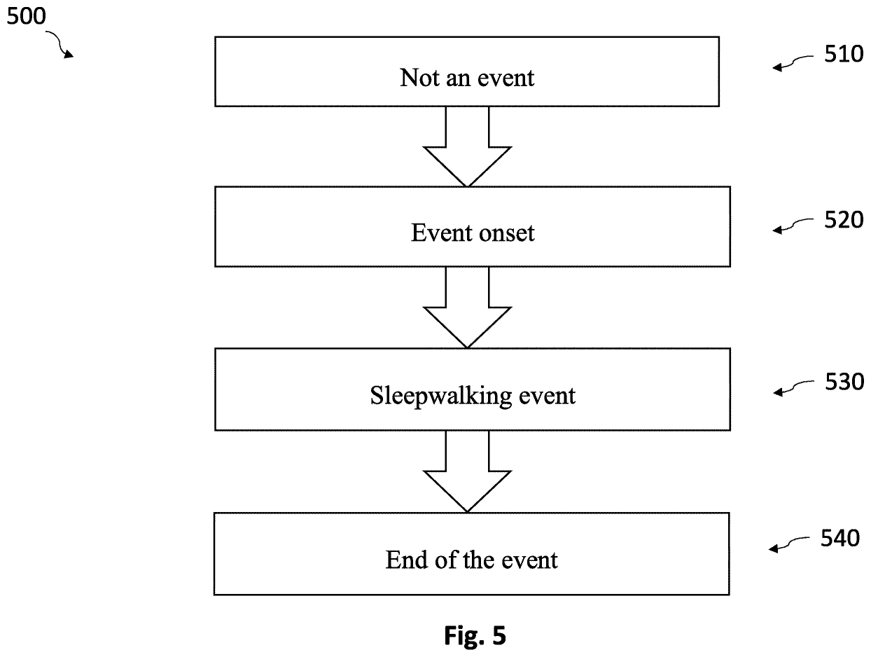

Based on the input of the movement and/or position measurements 314 and the sleeping physiological data 310, the apparatus 400 may be configured to detect specific stages of a somnambulism event, depicted in FIG. 5. The stages may include “not an event” 510, “event onset” 520, “sleepwalking event” 530, and/or “end of the event” 540. The phase “not an event” 510 may correspond to any time period determined to not be associated with a somnambulism event. Such a phase may include uninterrupted and undisturbed periods of sleep, which may include a cycling between NREM and REM sleep phases. The phase “event onset” 520 may correspond to the beginning of a somnambulism event. More specifically, it may correspond to a user learning upward from a resting position and supporting his own bodyweight, particularly during a deep sleep stage. The event onset may comprise a duration of seconds or minutes, with the user exhibiting certain behaviors and/or bodily functions that indicate that a somnambulism event has just begun. In such a phase, if the user's sleep is left undisturbed, the user is likely to begin sleepwalking. The phase “sleepwalking event” 530 may correspond to an instance where the user has previously shifted positions multiple times, supported his own bodyweight, and gotten up from a resting position, and is beginning to walk. The “sleepwalking event” 530 may continue as the user continues walking multiple steps in any direction. The duration of a “sleepwalking event” 530 may vary widely according to the user and a specific somnambulism event of the user. The “sleepwalking event” 530 may endure for a many seconds or many minutes, often lasting only a few minutes. The phase “end of the event” 540 may correspond to a period of seconds or minutes after a time period corresponding to a “sleepwalking event” 530 of the user. The “end of the event” 540 may correspond to weaker shifts in position, particularly in a resting position in which the shifts in position and movements of a “sleepwalking event” 530 are no longer possible.

The apparatus 400 is configured to detect the “event onset” 520 of a somnambulism event. By doing so, the apparatus may be configured to interrupt the sleep period quickly, directly at the onset of a somnambulism event. This may be desirable for a user that especially wishes to increase sleep quality, particularly if somnambulism events occur frequently for the user. Other users may wish to obtain a greater understanding of their personal somnambulism events and how often they may occur. To maintain safety during extended sleepwalking events, a feedback mechanism that is in communication with the apparatus 400 may be used.

Referring now back to FIG. 4, the processing circuitry 304 may be configured to prompt (cause) the feedback mechanism 360 to provide a vibratory and/or auditory feedback when the somnambulism event has entered the “event onset” 520 or, if preferred, the “sleepwalking event” 530. The feedback mechanism may be provided by a wearable device, such as the wearable device 312 or a second wearable device, or another device attached to the bed or mattress. The feedback mechanism may provide a vibration in a strong enough manner so that the user is awakened from a current sleep period. The vibration may also begin in a weaker form to prevent the user from being awakened and may become progressively stronger if the user continues to show signs of a somnambulism event after multiple weaker vibrations. The strength of vibration may be customized according to a user input 318 via the GUI 316.

The feedback mechanism may also provide auditory feedback to the user if the “event onset” 520 is detected. For example, a communicatively connected device with a digital speaker may be prompted by the processing circuitry 304 to provide a sound that is loud enough so that the user in the vicinity of the device is awakened from a current sleep period. The sound volume of the auditory feedback may also be lower at first to not awaken the user and then progressively increased if the user continues to show signs of a somnambulism event. Such a gradual increase in vibration or sound volume may minimize the intervention in the sleep period of the user. The vibratory and auditory feedback may be provided separately, simultaneously, or in consecutive fashion.

The feedback mechanism 360 may also be customized in providing the feedback based on a particular progression through the somnambulism event. For example, the user or a user's doctor may desire to better understand the somnambulism events that occur, but also to keep the user safe. The feedback mechanism 360 may be prompted to provide feedback to the user based on specific thresholds within the sleeping IMU data 310-4 and/or sleeping localization data 310-5. The feedback mechanism 360 may be prompted by a safety threshold, which may correspond to a threshold of motion or location. For example, the feedback mechanism 360 may be prompted to provide feedback to the user, given a movement that is quick enough or given that the user has traveled a minimum distance from a resting position used to fall asleep. Such a safety threshold may serve to keep the user safe by waking the user, if necessary. The use of such a safety threshold may also allow the somnambulism event to continue as long as the user is not vulnerable to an accident or injury. In this way, more sleeping physiological data 310 that corresponds to a somnambulism event may be collected, which may help to increase understanding of the user's sleeping disorder related to somnambulism and related behaviors.

For enabling a more precise detection the “event onset” 520 and the “sleepwalking event” 530, the processing circuitry 304 of the apparatus 400 may comprise one or more machine learning models. The machine learning models may include a sleep stage classification machine learning model 320, detection machine learning model 330, and a risk level score validation machine learning model 340.

The detection model 330 may communicate with the sleep stage classification model 320 to verify that the user is in a deep sleep state. Somnambulism events are known to occur in a deep sleep stage, partly because a user in a light sleep stage is unlikely to continue sleeping through sleepwalking motions. In general, a verification of the user being in a deep sleep stage may reduce the chances of a false positive detection of a somnambulism event, which would otherwise lead to unnecessarily disturbing the sleep period of the user. In particular, the sleep stage classification model 320 provides a focus of a certain portion of sleeping physiological data 310 only in the deep sleep stage. As such, attention-based learning solutions and temporal learning solutions, as previously described, may be further fine-tuned within a smaller and more appropriate pool of physiological data. In the unlikely event that the user proceeds to a “sleepwalking event” 530 without being in a deep sleep state or partially in a deep sleep state, the detection model 330 may communicate with the sleep stage classification model 320 to more broadly define the deep sleep stage for analyzing the sleeping physiological data 310. Such a customization toward the user for the detection model 330 and sleep stage classification model 320 may evolve as the user provides further iterations of sleeping physiological data 310.

The detection model 330 may also communicate with the risk level score validation model 340 to adjust one or more mechanisms for detection. For example, if the risk level score 120 of a somnambulism event occurring is relatively high, one or more thresholds of detection may be lowered. With lowered thresholds, the feedback mechanism 360 may intervene more quickly to prevent the user from proceeding from an “event onset” 520 to a “sleepwalking event”. The feedback mechanism 360 may also intervene more frequently. The feedback mechanism 360 may also be performed in a way that the user may maintains a state of deep sleep while being prevented from proceeding to the “sleepwalking event”. Such feedback mechanisms and methods related thereto may be improved through many periods of trial and error, tested on either the user one or more different users. The risk level score 120 and a finely tuned feedback mechanism may enable the user to sleep through somnambulism event occurrences. The risk level score 120 may also be used to prevent false positives. If the risk level score 120 is relatively low, then higher thresholds in the movement and/or position measurements 314 may be given to prevent the feedback mechanism 360 unnecessarily disturbing the sleep period of the user. The risk level score validation model 340 may be continually adjusted by receiving multiple risk level scores 120 and associated information.

In a further embodiment (not depicted), the physiological data 110 used to determine the risk level score 120 may be included with the risk level score 120. It may then be received by the interface circuitry 302 and the risk level score validation model 340. As such, the risk level score validation model 340 may receive the physiological data 110 generated before the sleep period of the user and provide such data in a processed form to the detection model 330. The detection model 330 may then more accurately predict a somnambulism event for the user. For example, the cardiac data 110-1 of the user recorded during active moments of the day may provide greater context for the sleeping cardiac data 310-1 recorded during the sleep period. Other datatypes of the physiological data 110 recorded during the day may also provide context for corresponding datatypes of the sleeping physiological data 310. Multiple iterations of sleeping physiological data 310, optionally with multiple iterations of physiological data 110 recorded while the user was active, may be analyzed be the three machine learning models 320;330;340 and may provide ground truth information for updating the models. For this, the machine learning models 320;330;340 may comprise multiple features previously described for the apparatus 200 to determine a risk level score in FIG. 2.

For example, each machine learning model may comprise an artificial neural network or representation encoder, such as a recurrent neural network (RNN) or a convolutional neural network (CNN). Each machine learning model may also apply attention mechanisms to selectively focus on specific parts of the sleeping physiological data 310, rather than processing the entire data at once. The attention mechanisms may apply, as previously described, soft attention, hard attention, multi-head attention mechanisms. Solutions based on transfer learning, elastic weight consolidation and prioritized experience memory may also be applied, as previously described.

The representational encoder may also apply temporal learning, online learning and/or continual learning solutions. While temporal learning and online and/or continual learning may not have as central of a role to a respective machine learning model for detection compared to the machine learning model 130 for determining a risk level score 120, they may aid in recognizing how a sleeping behavior of the user may evolve over multiple sleep cycles and multiple sleep periods. As such, the application of temporal learning, online learning and/or continual learning solutions may enable a better understanding of the somnambulism events and increase precision in the detection of the onset of a somnambulism event.

Such features related to machine learning models may be particularly useful in recognizing long-term patterns and relationships within multiple iterations of sleeping physiological data 310, as well as physiological data 110 recorded during active periods of the user. To enable saving multiple iterations of sleeping physiological data 310, the apparatus 400 may comprise a memory 306. The processing circuitry 304 may be configured to save in the memory 306 at least portions of the sleeping physiological data 310 of the user as a past sleeping physiological dataset 311 (past sleep-dataset 311). Each sleep period may lead to a saving of a corresponding past sleep-dataset 311, which may be added to a collection of past sleep-datasets already stored in the memory.

After a user has provided multiple saved past sleep-datasets 311, the machine learning models may be provided with ground truth information to update a respective training. The updated training may enable a more precise detection of a deep sleep state and a more precise detection of a somnambulism event of the specific user. An updated training with past sleep-datasets that do not correspond to a somnambulism event 311 may provide each of the machine learning models 320; 330; 340 with a predictable pattern of sleeping cardiac data 310-1, sleeping vascular data 310-2, and sleeping respiratory data 310-3. Such data may include typical average values and standard deviations. Such values may then provide a reliable basis of comparison for any deviation from such values during a somnambulism event. The updated training may further provide examples of outlying data corresponding to each datatype that does correspond to a somnambulism event.

The updated training may also enable the apparatus 400 to provide a more finely tuned feedback mechanism 360. For example, the thresholds related to the movement and/or position measurements, in particular thresholds for the accelerometer and/or gyroscope measurements, may be customized toward the user. This may be done through the risk level score validation model 340. The risk level score validation model 340 may receive multiple iterations of the risk level score 120, sleeping physiological data, and the occurrences of a somnambulism event. It may also monitor how frequently the feedback mechanism 360 has been applied for each somnambulism event. Such information may be used by the risk score level validation model to adjust thresholds related to the movement and/or position measurements to enable an appropriate degree of intervention when a somnambulism event occurs and also to prevent unnecessary disturbance of the sleep period. The risk score level validation model 340 may also receive user input 318 via the GUI 316 that the feedback mechanisms are too frequent or do intervene soon enough and further customize the thresholds accordingly.

The interface circuitry 302 may also be configured to receive past sleep-datasets 311 as a user input 318 via the GUI 316. This may be particularly useful to import past sleep-datasets corresponding to sleep periods of one or more different users and quickly expand a pool of data to be used as ground truth information. For example, such past sleep-datasets may also be quickly made available and received by means of a data-sharing system. Past sleep-datasets of multiple users who are using the same apparatus or another apparatus comprising similar features may be saved and shared with a central server, preferably anonymously. This may enable participating users to build a pool of available ground truth information more quickly. The machine learning models can then be customized for the user gradually over time by further iterations of the user's past sleep-datasets 311.

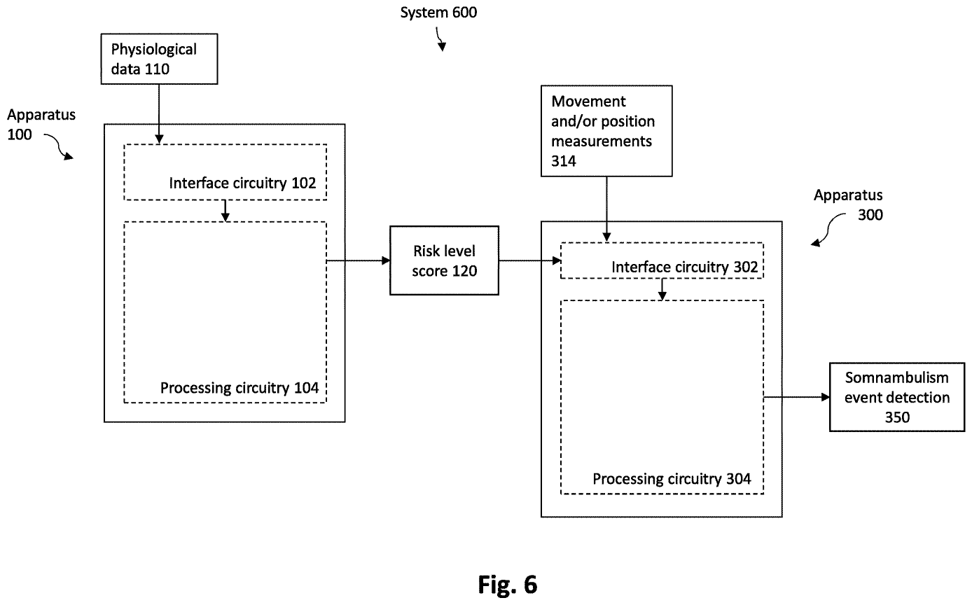

Each past sleep-dataset 311 may also be exported to an external device, such as the apparatus 100; 200 configured to determine the risk level score 120. The apparatus 100; 200 for determining the risk level score 120 may be provided with further ground truth information for a more precise risk level score determination that is customized toward the user. Further details related thereto will be given in the following with reference to FIG. 6.

FIG. 6 schematically illustrates a system 600 to demonstrate how the apparatus 100 of FIG. 1 for determining a somnambulism event risk level score of a user (which will be referred to as the risk-level-score apparatus 100) may complement the features of the apparatus 300 of FIG. 3 for detecting the onset of a somnambulism event (which will be referred to as the detection apparatus 300).

The risk level score 120 generated by the risk-level-score apparatus 100 may be generated and then be provided as an input to the detection apparatus 300. The risk-level score 120 may provide information that may be used to improve a precision of detection by the detection apparatus 300. In particular, one or more thresholds related to the movement and/or position measurements 314 may be altered depending on whether the risk level score is high or low, as described in FIG. 4. Thresholds may also be set within other forms of physiological data, such as cardiac data, vascular data, respiratory data, IMU data, and/or localization data, as previously described, and the risk level score may aid in adjusting the thresholds to a more finely tuned value according to the user. Such data may enable a more precise detection of whether the user is in a deep sleep state, which may be a condition for the detection apparatus 300 to detect a somnambulism event of the user.

Furthermore, the physiological data 110 used for determining the risk level score 120 may also be provided to the detection apparatus 300 (not depicted). In a particular embodiment, the risk-level-score apparatus 100 may comprise a machine learning model, as previously described. The detection apparatus 300 may also comprise one or more machine learning models. Physiological data 110 provided as input to the detection apparatus 300 may enable a greater customization of the machine learning models toward the user. The processing circuitry 104 of the risk-level-score apparatus 100 may also provide the physiological data 110 in a processed form with the risk level score 120, so that it may be easily incorporated into the machine learning models of the detection apparatus 300. In a particular embodiment, an input of processed physiological data output with the risk level score 120 by the risk-level-score apparatus 100 may be used as ground truth information to improve a training of one or more machine learning models of the detection apparatus 300.

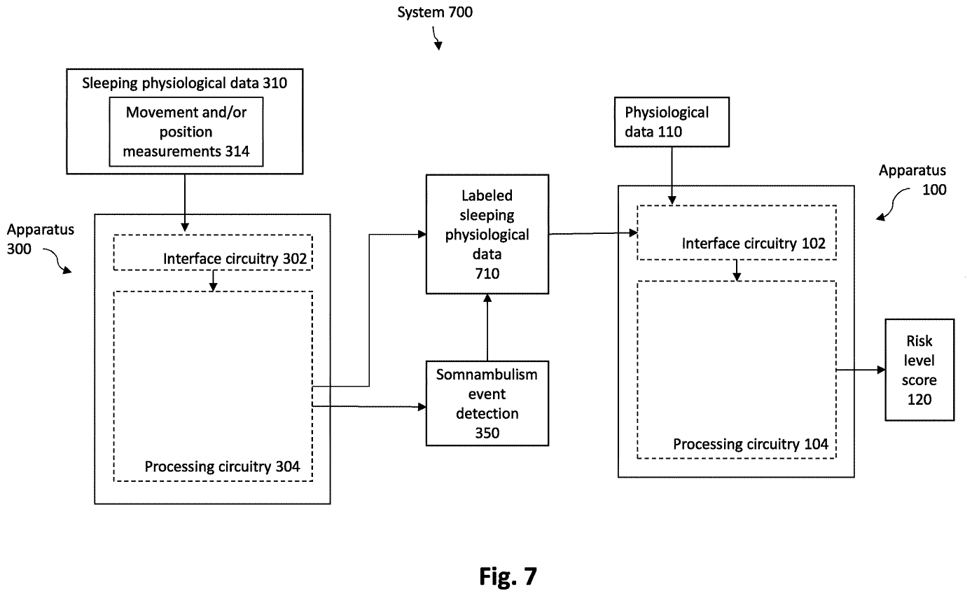

In addition to the risk-level-score apparatus 100 complementing the features of the detection apparatus 300, the detection apparatus 300 may also complement the features of the risk-level-score apparatus 100. Such a system is provided in FIG. 7.

FIG. 7 schematically illustrates a system 700 with the detection apparatus 300 of FIG. 1 providing input to the risk-level-score apparatus 100 of FIG. 3.

In the system 700, the movement and/or position measurements 314 are additionally part of a collection of sleeping physiological data 310 measured and recorded while the user was sleeping. The sleeping physiological data 310 may have been measured, recorded, and then transmitted from a wearable device to the interface circuitry 302, as described in FIG. 4. After being received by the interface circuitry 302, the sleeping physiological data 310, including the movement and/or position measurements 314, is used to detect the onset of a somnambulism event whenever it may occur. When a sleep period is over, the sleeping physiological data may be output as labeled sleeping physiological data 710.

The labeled sleeping physiological data 710 comprises an associated label of whether or not it corresponds to a somnambulism event occurrence. In the case of a somnambulism event detection 350, the processing circuitry 304 is configured to generate a label to indicate that the sleeping physiological data 310 received during the sleep period corresponds to a somnambulism event. If no somnambulism event was detected, the processing circuitry 304 may generate the labeled sleeping physiological dataset with an indication of not corresponding to a somnambulism event. Both forms of labeled sleeping physiological data 710 are depicted in FIG. 7 to be provided as input to the interface circuity 102 of the risk-level-score apparatus. The labeled sleeping physiological data 710 may complement the physiological data 110, which includes measurements recorded while the user was awake and active. The labeled sleeping physiological data 710 may provide measurements of bodily functions of the user while sleeping, while the physiological data 110 may provide such measurements while the user is awake and active. Thus, both datasets 110; 710 may correspond to different portions of the same extended time period before a sleep period. Furthermore, data reflecting unique characteristics of the user's bodily functions or data reflecting stress in a user's lived experiences within the time period may be expressed in both datasets 110; 710. An input of both complementary datasets 110; 710 measured during different portions within the same extended time span (such as multiple weeks or months) may enable the risk-level-score apparatus to better customize one or more a machine learning models towards the user, as previously described in FIG. 2.

As such the system of the two apparatuses working together may improve a precision in the determination of the risk level score 120 to a degree that is otherwise not possible. In fact, the two systems 600 and 700 may work simultaneously to enhance the functions of both the apparatuses 100 and 300.

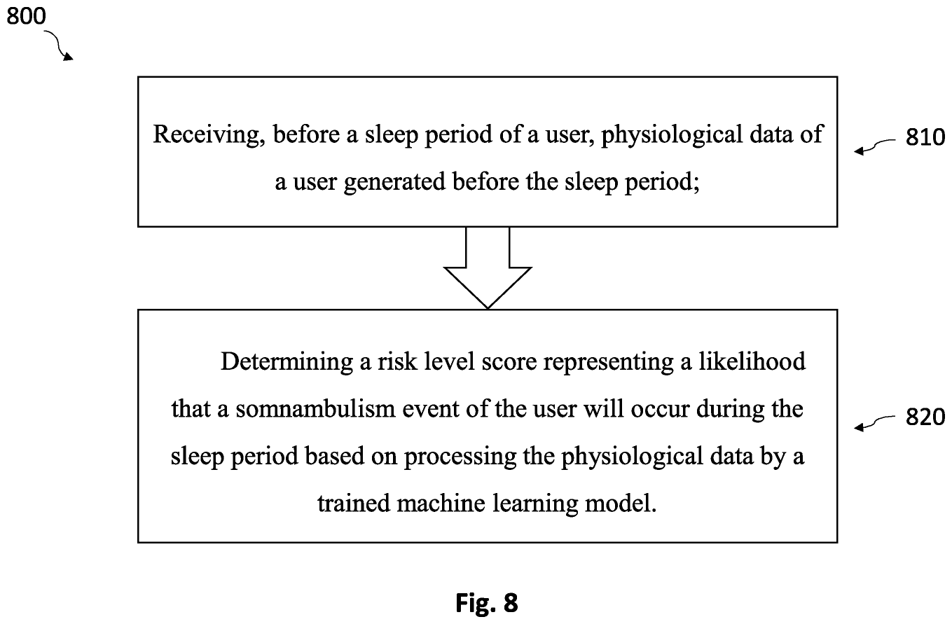

FIG. 8 illustrates a flow chart of an exemplary method for determining a somnambulism event risk level score of a user.

The method 800 comprises receiving 810, before a sleep period of a user, physiological data of the user generated before the sleep period. The method further comprises determining 820 a risk level score representing a likelihood that a somnambulism event will occur for the user during the sleep period based on processing the physiological data by a trained machine learning model.

The method 800 may optionally comprise one or more further features described in FIG. 2. For example, the method 800 may comprise receiving physiological data from a wearable device, wherein the physiological data comprises cardiac, respiratory, vascular, inertia measurement unit, and/or localization data of the user. The method 800 may further comprise measuring physiological data over a pre-specified time period before the sleep period that is adjustable according to a user input and/or a threshold within the physiological data of the user. Further, the method 800 may comprise matching patterns between the physiological data of the user and previous physiological data corresponding to previous somnambulism events of the user and/or one or more different users.

The method 800 may further comprise aspects related to a machine training model. For instance, the method 800 may comprise training a machine learning model with ground truth information that includes previous physiological data of the user and/or one or more different users corresponding to a previous somnambulism event occurrence of the respective user. The method 800 may further comprise updating the training the machine learning model based on further iterations of previous physiological data of the user and/or one or more different users. The method 800 may further comprise implementing temporal representation learning on the ground truth information for one or more pre-specified time periods before each respective sleep period of each respective user. Finally, the method 800 may comprise receiving a notification of whether or not a somnambulism event occurred during the sleep period and updating the training of the machine learning model with a new iteration of ground truth information. The ground truth information may comprise physiological data of the user generated before the sleep period, the corresponding risk level score, and a corresponding label of whether or not a somnambulism event occurred during the sleep period.

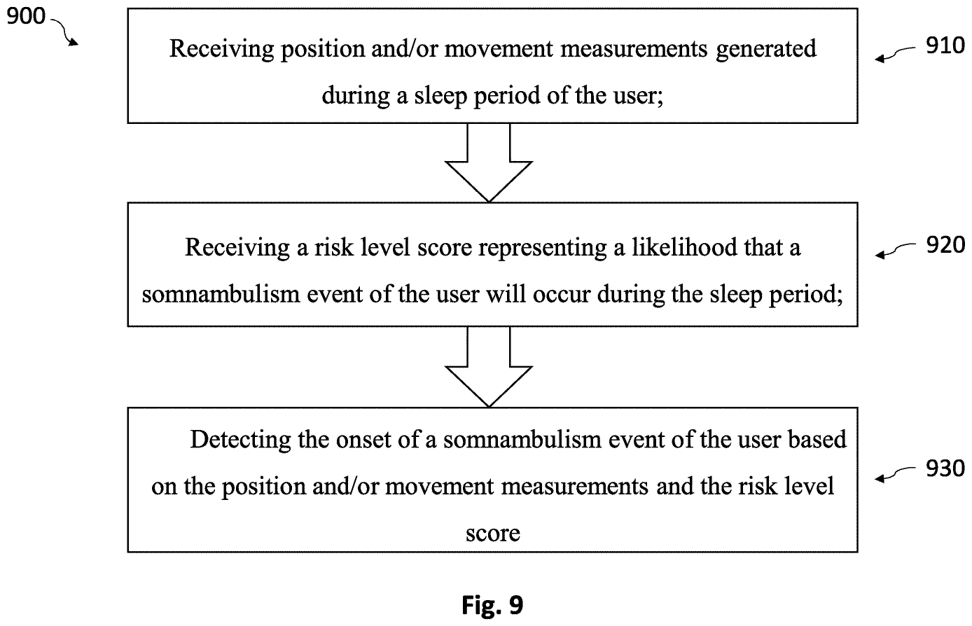

FIG. 9 illustrates a flow chart of an exemplary method 900 for detecting the onset of a somnambulism event of a user during a sleep period of the user.

The method comprises receiving 910 movement and/or position measurements of the user generated during the sleep period of the user. The method further comprises receiving 920 a risk level score representing a likelihood that a somnambulism event will occur for the user during the sleep period of the user. In addition, the method further comprises detecting 930 the onset of a somnambulism event of the user based on the movement and/or position measurements and the risk level score.

The method 900 may optionally comprise one or more further features described in FIG. 4. For example, the method 900 may comprise detecting the onset of a somnambulism event of the user based on physiological data generated both before and during the sleep period. Furthermore, the method 900 may comprise detecting the onset of a somnambulism event of the user based on one or more thresholds within the movement and/or position measurements that are customized according to the user and/or the risk level score. The method 900 may further comprise determining a sleep stage of the user during the sleep period and detecting a somnambulism event of the user further on the condition that the user has transitioned to the sleep stage. The method 900 may further comprise identifying transitions between somnambulism event stages, which may include not an event, event onset, sleepwalking event and/or end of the event. The method 900 may further comprise causing a somnambulism feedback device to provide vibratory and/or auditory feedback to the user if a somnambulism event is detected. The method 900 may also further comprise saving in a memory and/or exporting to an external device at least part of the movement and/or position measurements of the sleep period as part of a sleep-dataset.

The method 900 may further comprise aspects related to a machine learning model. For example, the method 900 may comprise training a machine learning model by ground truth information comprising respective sleep-datasets of the user and/or one or more different users, each sleep-dataset having associated therewith a corresponding label of whether or not a somnambulism event occurred during the sleep period. The method 900 may further comprise applying attention-based machine learning and/or continual machine learning techniques on the physiological data of the respective sleep-datasets.

The following examples pertain to further embodiments:
(1) An apparatus for determining a somnambulism event risk level score of a user, the apparatus comprising interface circuitry configured to receive, before a sleep period of the user, physiological data of the user generated before the sleep period, and processing circuitry configured to determine a risk level score representing a likelihood that a somnambulism event of the user will occur during the sleep period based on the physiological data generated before the sleep period.
(2) The apparatus of (1), wherein the interface circuitry is communicatively connectable to a wearable device and is configured to receive the physiological data from the wearable device, wherein the physiological data comprises one or more of cardiac, respiratory, vascular, inertia measurement unit, and localization data of the user.
(3) The apparatus of (1) or (2), wherein the physiological data is measured during a pre-specified time period before the sleep period, wherein the pre-specified time period is adjustable based on a user input and/or one or more thresholds within the physiological data of the user.
(4) The apparatus of any one of (1) to (3), wherein the processing circuitry is configured to match patterns between the physiological data of the user and previous physiological data corresponding to previous somnambulism event occurrences of the user and/or one or more different users.
(5). The apparatus of any one of (1) to (4), wherein the processing circuitry comprises a machine learning model, the machine learning model trained and periodically updated by ground truth information comprising previous physiological data of the user and/or one or more different users corresponding to a previous somnambulism event occurrence of the respective user.
(6) The apparatus of (5), wherein the processing circuitry is configured to update the training of the machine learning model based on further iterations of previous physiological data of the user and/or one or more different users.
(7) The apparatus of (5) or (6), wherein the machine learning model is configured to implement temporal representation learning on the ground truth information for one or more pre-specified time periods before each respective sleep period of each respective user.
(8) The apparatus of any one of (5) to (7), wherein the interface circuitry is configured to receive a notification after the sleep period of whether or not a somnambulism event occurred during the sleep period, and wherein the processing circuitry is configured to update the training of the machine learning model with a new iteration of ground truth information comprising the physiological data of the user generated before the sleep period, the corresponding risk level score, and a corresponding label of whether or not a somnambulism event occurred during the sleep period.
(9) A method for determining a somnambulism event risk level score of a user, the method comprising receiving, before a sleep period of the user, physiological data of the user generated before the sleep period, and determining a risk level score representing a likelihood that a somnambulism event of the user will occur during the sleep period based on processing the physiological data by a trained machine learning model.
(10) An apparatus for detecting the onset of a somnambulism event of a user during a sleep period of the user, the apparatus comprising interface circuitry configured to receive movement and/or position measurements of the user generated during the sleep period of the user, and a risk level score representing a likelihood that a somnambulism event will occur for the user during the sleep period; and processing circuitry configured to detect the onset of a somnambulism event of the user based on the physiological data generated during the sleep period and the risk level score.
(11) The apparatus of (10), wherein the processing circuitry is configured to detect the onset of a somnambulism event of the user based on one or more thresholds within the movement and/or position measurements, wherein the one or more thresholds are customized according to the user and/or the risk level score.
(12) The apparatus of (10) or (11), wherein the processing circuitry is configured to determine a sleep stage of the user during the sleep period, including light sleep, deep sleep, and/or rapid eye movement (REM) sleep, and configured to detect the onset of a somnambulism event of the user further on the condition that user has transitioned to the sleep stage.
(13) The apparatus of any one of (10) to (12), wherein the processing circuitry is configured to further identify transitions between somnambulism event stages, including not an event, event onset, sleepwalking event, and/or end of the event.
(14) The apparatus of any one of (10) to (13), wherein an output of the processing circuitry is communicatively connectable to a somnambulism feedback device and the processing circuitry is configured to cause the somnambulism feedback device to provide vibratory and/or auditory feedback to the user if the onset of a somnambulism event is detected.
(15) The apparatus of any one of (10) to (14), wherein the interface circuitry is configured to receive physiological data of the user generated during the sleep period of the user, and wherein
the processing circuitry is configured to detect the onset of a somnambulism event of the user further based on the physiological data generated during the sleep period.
(16) The apparatus of (15), wherein the interface circuitry is configured to receive physiological data generated before the sleep period of the user that was used to determine the risk level score, and wherein the processing circuitry is configured to detect the onset of a somnambulism event of the user based on the physiological data generated both before and during the sleep period.
(17) The apparatus of (15) or (16), wherein the interface circuitry is communicatively connectable to a wearable device and is configured to receive the physiological data from the wearable device, wherein the physiological data further comprises cardiac, respiratory, vascular, inertia measurement unit, and/or localization data of the user generated during the sleep period.
(18) The apparatus of any one of (15) to (17), the apparatus further comprising a memory, and wherein the processing circuitry is configured to save in the memory and/or export to an external device at least part of the physiological data of the user generated during the sleep period as part of a sleep-dataset.
(19) The apparatus of (18), wherein the processing circuitry is based on a machine learning model trained by ground truth information, the ground truth information comprising respective sleep-datasets of the user and/or one or more different users, each sleep-dataset having associated therewith a corresponding label of whether or not a somnambulism event occurred during the sleep period.
(20) The apparatus of (19), wherein the machine learning model comprises a neural network architecture and applies attention-based machine learning and/or continual machine learning on at least a portion of the physiological data of one or more respective sleep-datasets.
(21) A method for detecting the onset of a somnambulism event of a user during a sleep period of the user, the method comprising receiving movement and/or position measurements of the user generated during the sleep period of the user, receiving a risk level score representing a likelihood that a somnambulism event of the user will occur during the sleep period; and detecting the onset of a somnambulism event of the user based on the movement and/or position measurements and the risk level score.
(22) A system for detecting the onset of a somnambulism event of a user during a sleep period of the user, the system comprising
a first apparatus for determining a somnambulism event risk level score of a user, the first apparatus comprising:
   interface circuitry configured to receive, before the sleep period, physiological data of the user generated before the sleep period and
   processing circuitry configured to determine a risk level score representing a likelihood that a somnambulism event of the user will occur during the sleep period based on the physiological data; and
a second apparatus for detecting the onset of a somnambulism event of the user during the sleep period, the second apparatus comprising:
   interface circuitry configured to receive movement and/or position measurements of the user generated during the sleep period and to receive the risk level score from the first apparatus; and
   processing circuitry configured to detect the onset of a somnambulism event of the user based on the movement and/or position measurements and the risk level score.
(23) A system for determining a somnambulism event risk level score of a user before a sleep period, the system comprising
a first apparatus for detecting the onset of a somnambulism event of the user while the user was sleeping, the first apparatus comprising:
   interface circuitry configured to receive sleeping physiological data of the user generated while the user was sleeping, the sleeping physiological data including movement and/or position measurements; and
   processing circuitry configured to detect the onset of a somnambulism event of the user based on the sleeping physiological data; and
a second apparatus for determining a somnambulism event risk level score of a user, the second apparatus comprising:
   interface circuitry configured to receive physiological data of the user generated while the user was awake, and, from the first apparatus, labeled sleeping physiological data of the user generated while the user was sleeping and comprising a label of whether or not the sleeping physiological corresponds to a somnambulism event, and
processing circuitry configured to determine a risk level score representing a likelihood that a somnambulism event of the user will occur during the sleep period based on the physiological data generated while the user was awake and the sleeping physiological data.

The aspects and features described in relation to a particular one of the previous examples may also be combined with one or more of the further examples to replace an identical or similar feature of that further example or to additionally introduce the features into the further example.

Examples may further be or relate to a (computer) program including a program code to execute one or more of the above methods when the program is executed on a computer, processor or other programmable hardware component. Thus, steps, operations or processes of different ones of the methods described above may also be executed by programmed computers, processors or other programmable hardware components. Examples may also cover program storage devices, such as digital data storage media, which are machine-, processor- or computer-readable and encode and/or contain machine-executable, processor-executable or computer-executable programs and instructions. Program storage devices may include or be digital storage devices, magnetic storage media such as magnetic disks and magnetic tapes, hard disk drives, or optically readable digital data storage media, for example. Other examples may also include computers, processors, control units, (field) programmable logic arrays ((F)PLAs), (field) programmable gate arrays ((F)PGAs), graphics processor units (GPU), application-specific integrated circuits (ASICs), integrated circuits (ICs) or system-on-a-chip (SoCs) systems programmed to execute the steps of the methods described above.

Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the present disclosure can be implemented in hardware or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the present disclosure comprise a data carrier having electronically readable control signals, which are capable of cooperating with a programmable computer system, such that one of the methods described herein is performed.

Generally, embodiments of the present present disclosure can be implemented as a computer program product with a program code, the program code being operative for performing one of the methods when the computer program product runs on a computer. The program code may, for example, be stored on a machine readable carrier.

Other embodiments comprise the computer program for performing one of the methods described herein, stored on a machine readable carrier.

In other words, an embodiment of the present present disclosure is, therefore, a computer program having a program code for performing one of the methods described herein, when the computer program runs on a computer.

A further embodiment of the present present disclosure is, therefore, a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present present disclosure is an apparatus as described herein comprising a processor and the storage medium.

A further embodiment of the present disclosure is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to, perform one of the methods described herein.

A further embodiment comprises a computer having installed thereon the computer program for performing one of the methods described herein.

A further embodiment according to the present disclosure comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

Embodiments may be based on using a machine-learning model or machine-learning algorithm. Machine learning may refer to algorithms and statistical models that computer systems may use to perform a specific task without using explicit instructions, instead relying on models and inference. For example, in machine-learning, instead of a rule-based transformation of data, a transformation of data may be used, that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and/or training sequences (e.g. words or sentences) and associated training content information (e.g. labels or annotations), the machine-learning model “learns” to recognize the content of the images, so the content of images that are not included in the training data can be recognized using the machine-learning model. The same principle may be used for other kinds of sensor data as well: By training a machine-learning model using training sensor data and a desired output, the machine-learning model “learns” a transformation between the sensor data and the output, which can be used to provide an output based on non-training sensor data provided to the machine-learning model. The provided data (e.g. sensor data, meta data and/or image data) may be preprocessed to obtain a feature vector, which is used as input to the machine-learning model.

Machine-learning models may be trained using training input data. The examples specified above use a training method called “supervised learning”. In supervised learning, the machine-learning model is trained using a plurality of training samples, wherein each sample may comprise a plurality of input data values, and a plurality of desired output values, i.e. each training sample is associated with a desired output value. By specifying both training samples and desired output values, the machine-learning model “learns” which output value to provide based on an input sample that is similar to the samples provided during the training. Apart from supervised learning, semi-supervised learning may be used. In semi-supervised learning, some of the training samples lack a corresponding desired output value. Supervised learning may be based on a supervised learning algorithm (e.g. a classification algorithm, a regression algorithm or a similarity learning algorithm. Classification algorithms may be used when the outputs are restricted to a limited set of values (categorical variables), i.e. the input is classified to one of the limited set of values. Regression algorithms may be used when the outputs may have any numerical value (within a range). Similarity learning algorithms may be similar to both classification and regression algorithms but are based on learning from examples using a similarity function that measures how similar or related two objects are. Apart from supervised or semi-supervised learning, unsupervised learning may be used to train the machine-learning model. In unsupervised learning, (only) input data might be supplied and an unsupervised learning algorithm may be used to find structure in the input data (e.g. by grouping or clustering the input data, finding commonalities in the data). Clustering is the assignment of input data comprising a plurality of input values into subsets (clusters) so that input values within the same cluster are similar according to one or more (pre-defined) similarity criteria, while being dissimilar to input values that are included in other clusters.

Reinforcement learning is a third group of machine-learning algorithms. In other words, reinforcement learning may be used to train the machine-learning model. In reinforcement learning, one or more software actors (called “software agents”) are trained to take actions in an environment. Based on the taken actions, a reward is calculated. Reinforcement learning is based on training the one or more software agents to choose the actions such, that the cumulative reward is increased, leading to software agents that become better at the task they are given (as evidenced by increasing rewards).

Furthermore, some techniques may be applied to some of the machine-learning algorithms. For example, feature learning may be used. In other words, the machine-learning model may at least partially be trained using feature learning, and/or the machine-learning algorithm may comprise a feature learning component. Feature learning algorithms, which may be called representation learning algorithms, may preserve the information in their input but also transform it in a way that makes it useful, often as a pre-processing step before performing classification or predictions. Feature learning may be based on principal components analysis or cluster analysis, for example.

In some examples, anomaly detection (i.e. outlier detection) may be used, which is aimed at providing an identification of input values that raise suspicions by differing significantly from the majority of input or training data. In other words, the machine-learning model may at least partially be trained using anomaly detection, and/or the machine-learning algorithm may comprise an anomaly detection component.

In some examples, the machine-learning algorithm may use a decision tree as a predictive model. In other words, the machine-learning model may be based on a decision tree. In a decision tree, observations about an item (e.g. a set of input values) may be represented by the branches of the decision tree, and an output value corresponding to the item may be represented by the leaves of the decision tree. Decision trees may support both discrete values and continuous values as output values. If discrete values are used, the decision tree may be denoted a classification tree, if continuous values are used, the decision tree may be denoted a regression tree.

Association rules are a further technique that may be used in machine-learning algorithms. In other words, the machine-learning model may be based on one or more association rules. Association rules are created by identifying relationships between variables in large amounts of data. The machine-learning algorithm may identify and/or utilize one or more relational rules that represent the knowledge that is derived from the data. The rules may e.g. be used to store, manipulate or apply the knowledge.

Machine-learning algorithms are usually based on a machine-learning model. In other words, the term “machine-learning algorithm” may denote a set of instructions that may be used to create, train or use a machine-learning model. The term “machine-learning model” may denote a data structure and/or set of rules that represents the learned knowledge (e.g. based on the training performed by the machine-learning algorithm). In embodiments, the usage of a machine-learning algorithm may imply the usage of an underlying machine-learning model (or of a plurality of underlying machine-learning models). The usage of a machine-learning model may imply that the machine-learning model and/or the data structure/set of rules that is the machine-learning model is trained by a machine-learning algorithm.

For example, the machine-learning model may be an artificial neural network (ANN). ANNs are systems that are inspired by biological neural networks, such as can be found in a retina or a brain. ANNs comprise a plurality of interconnected nodes and a plurality of connections, so-called edges, between the nodes. There are usually three types of nodes, input nodes that receiving input values, hidden nodes that are (only) connected to other nodes, and output nodes that provide output values. Each node may represent an artificial neuron. Each edge may transmit information, from one node to another. The output of a node may be defined as a (non-linear) function of its inputs (e.g. of the sum of its inputs). The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e. to achieve a desired output for a given input. Alternatively, the machine-learning model may be a support vector machine, a random forest model or a gradient boosting model. Support vector machines (i.e. support vector networks) are supervised learning models with associated learning algorithms that may be used to analyze data (e.g. in classification or regression analysis). Support vector machines may be trained by providing an input with a plurality of training input values that belong to one of two categories. The support vector machine may be trained to assign a new input value to one of the two categories. Alternatively, the machine-learning model may be a Bayesian network, which is a probabilistic directed acyclic graphical model. A Bayesian network may represent a set of random variables and their conditional dependencies using a directed acyclic graph. Alternatively, the machine-learning model may be based on a genetic algorithm, which is a search algorithm and heuristic technique that mimics the process of natural selection.

It is further understood that the disclosure of several steps, processes, operations or functions disclosed in the description or claims shall not be construed to imply that these operations are necessarily dependent on the order described, unless explicitly stated in the individual case or necessary for technical reasons. Therefore, the previous description does not limit the execution of several steps or functions to a certain order. Furthermore, in further examples, a single step, function, process or operation may include and/or be broken up into several sub-steps, -functions, -processes or -operations.

If some aspects have been described in relation to a device or system, these aspects should also be understood as a description of the corresponding method. For example, a block, device or functional aspect of the device or system may correspond to a feature, such as a method step, of the corresponding method. Accordingly, aspects described in relation to a method shall also be understood as a description of a corresponding block, a corresponding element, a property or a functional feature of a corresponding device or a corresponding system.

The following claims are hereby incorporated in the detailed description, wherein each claim may stand on its own as a separate example. It should also be noted that although in the claims a dependent claim refers to a particular combination with one or more other claims, other examples may also include a combination of the dependent claim with the subject matter of any other dependent or independent claim. Such combinations are hereby explicitly proposed, unless it is stated in the individual case that a particular combination is not intended. Furthermore, features of a claim should also be included for any other independent claim, even if that claim is not directly defined as dependent on that other independent claim.

1. An apparatus for determining a somnambulism event risk level score of a user, the apparatus comprising:
interface circuitry configured to receive, before a sleep period of the user, physiological data of the user generated before the sleep period, and
processing circuitry configured to determine a risk level score representing a likelihood that a somnambulism event of the user will occur during the sleep period based on the physiological data generated before the sleep period.

2. The apparatus of claim 1, wherein
the interface circuitry is communicatively connectable to a wearable device and is configured to receive the physiological data from the wearable device, wherein the physiological data comprises one or more of cardiac, respiratory, vascular, inertia measurement unit, and localization data of the user.

3. The apparatus of claim 1, wherein
the physiological data is measured during a pre-specified time period before the sleep period, wherein the pre-specified time period is adjustable based on a user input and/or one or more thresholds within the physiological data of the user.

4. The apparatus of claim 1, wherein
the processing circuitry is configured to match patterns between the physiological data of the user and previous physiological data corresponding to previous somnambulism event occurrences of the user and/or one or more different users.

5. The apparatus of claim 1, wherein
the processing circuitry comprises a machine learning model, the machine learning model trained and periodically updated by ground truth information comprising previous physiological data of the user and/or one or more different users corresponding to a previous somnambulism event occurrence of the respective user.

6. The apparatus of claim 5, wherein
the interface circuitry is configured to receive a notification after the sleep period of whether or not a somnambulism event occurred during the sleep period, and wherein
the processing circuitry is configured to update the training of the machine learning model with a new iteration of ground truth information comprising the physiological data of the user generated before the sleep period, the corresponding risk level score, and a corresponding label of whether or not a somnambulism event occurred during the sleep period.

7. A method for determining a somnambulism event risk level score of a user, the method comprising:
receiving, before a sleep period of the user, physiological data of the user generated before the sleep period, and
determining a risk level score representing a likelihood that a somnambulism event of the user will occur during the sleep period based on processing the physiological data by a trained machine learning model.

8. An apparatus for detecting the onset of a somnambulism event of a user during a sleep period of the user, the apparatus comprising:
interface circuitry configured to receive
movement and/or position measurements of the user generated during the sleep period of the user, and
a risk level score representing a likelihood that a somnambulism event will occur for the user during the sleep period; and
processing circuitry configured to detect the onset of a somnambulism event of the user based on the physiological data generated during the sleep period and the risk level score.

9. The apparatus of claim 8, wherein
the processing circuitry is configured to detect the onset of a somnambulism event of the user based on one or more thresholds within the movement and/or position measurements, wherein the one or more thresholds are customized according to the user and/or the risk level score.

10. The apparatus of claim 8, wherein
the processing circuitry is configured to determine a sleep stage of the user during the sleep period, including light sleep, deep sleep, and/or rapid eye movement (REM) sleep, and configured to detect the onset of a somnambulism event of the user further on the condition that user has transitioned to the sleep stage.

11. The apparatus of claim 8, wherein
the processing circuitry is configured to further identify transitions between somnambulism event stages, including not an event, event onset, sleepwalking event, and/or end of the event.

12. The apparatus of claim 8, wherein
an output of the processing circuitry is communicatively connectable to a somnambulism feedback device and the processing circuitry is configured to cause the somnambulism feedback device to provide vibratory and/or auditory feedback to the user if the onset of a somnambulism event is detected.

13. The apparatus of claim 8, wherein
the interface circuitry is configured to receive physiological data of the user generated during the sleep period of the user, and wherein
the processing circuitry is configured to detect the onset of a somnambulism event of the user further based on the physiological data generated during the sleep period.

14. The apparatus of claim 13, wherein
the interface circuitry is configured to receive physiological data generated before the sleep period of the user that was used to determine the risk level score, and wherein
the processing circuitry is configured to detect the onset of a somnambulism event of the user based on the physiological data generated both before and during the sleep period.

15. The apparatus of claim 13, wherein
the interface circuitry is communicatively connectable to a wearable device and is configured to receive the physiological data from the wearable device, wherein the physiological data further comprises cardiac, respiratory, vascular, inertia measurement unit, and/or localization data of the user generated during the sleep period.

16. The apparatus of claim 13, the apparatus further comprising a memory, and wherein the processing circuitry is configured to save in the memory and/or export to an external device at least part of the physiological data of the user generated during the sleep period as part of a sleep-dataset.

17. The apparatus of claim 16, wherein
the processing circuitry is based on a machine learning model trained by ground truth information, the ground truth information comprising respective sleep-datasets of the user and/or one or more different users, each sleep-dataset having associated therewith a corresponding label of whether or not a somnambulism event occurred during the sleep period.

18. A method for detecting the onset of a somnambulism event of a user during a sleep period of the user, the method comprising:
receiving movement and/or position measurements of the user generated during the sleep period of the user;
receiving a risk level score representing a likelihood that a somnambulism event of the user will occur during the sleep period; and
detecting the onset of a somnambulism event of the user based on the movement and/or position measurements and the risk level score.

19. A system for detecting the onset of a somnambulism event of a user during a sleep period of the user, the system comprising
a first apparatus for determining a somnambulism event risk level score of a user, the first apparatus comprising:
interface circuitry configured to receive, before the sleep period, physiological data of the user generated before the sleep period and
processing circuitry configured to determine a risk level score representing a likelihood that a somnambulism event of the user will occur during the sleep period based on the physiological data; and
a second apparatus for detecting the onset of a somnambulism event of the user during the sleep period, the second apparatus comprising:
interface circuitry configured to receive movement and/or position measurements of the user generated during the sleep period and to receive the risk level score from the first apparatus; and
processing circuitry configured to detect the onset of a somnambulism event of the user based on the movement and/or position measurements and the risk level score.

20. A system for determining a somnambulism event risk level score of a user before a sleep period, the system comprising
a first apparatus for detecting the onset of a somnambulism event of the user while the user was sleeping, the first apparatus comprising:
interface circuitry configured to receive sleeping physiological data of the user generated while the user was sleeping, the sleeping physiological data including movement and/or position measurements; and
processing circuitry configured to detect the onset of a somnambulism event of the user based on the sleeping physiological data; and
a second apparatus for determining a somnambulism event risk level score of a user, the second apparatus comprising:
interface circuitry configured to receive physiological data of the user generated while the user was awake, and, from the first apparatus, labeled sleeping physiological data of the user generated while the user was sleeping and comprising a label of whether or not the sleeping physiological corresponds to a somnambulism event, and
processing circuitry configured to determine a risk level score representing a likelihood that a somnambulism event of the user will occur during the sleep period based on the physiological data generated while the user was awake and the sleeping physiological data.
